(19) United States
(12) Patent Application Publication
(10) Pub. No.: US 2013/0030238 A1
(43) Pub. Date: Jan. 31, 2013

(54) BRACHYTHERAPY DEVICES, KITS AND METHODS OF USE

(71) Applicants: Linda A. Kelley, Los Gatos, CA (US); Randall W. Holt, Chico, CA (US); Aileen Lum, Menlo Park, CA (US); Anuradha Walawalkar, Sunnyvale, CA (US); Thomas W. Rusch, Hopkins, MN (US)

(72) Inventors: Linda A. Kelley, Los Gatos, CA (US); Randall W. Holt, Chico, CA (US); Aileen Lum, Menlo Park, CA (US); Anuradha Walawalkar, Sunnyvale, CA (US); Thomas W. Rusch, Hopkins, MN (US)

(21) Appl. No.: 13/192,426

(22) Filed: Jul. 27, 2011

(51) Int. Cl. A61N 5/10; A61N 5/00

(52) U.S. Cl. 600/6; 600/3

(57) ABSTRACT

The radial dose function of an electronic x-ray brachytherapy source is flattened by filtering with transition metals in the fourth row of the periodic table, i.e. titanium through nickel. Titanium-walled applicator devices of small diameter, under 10 mm, and with wall thicknesses of about 0.2 mm to 0.6 mm, are disclosed. The walls can be of titanium or alloys thereof, providing adequate strength and flattening the radial dose function curve particularly for x-rays in an energy range of about 45 kV to 55 kV.

### BACKGROUND OF THE INVENTION

The present application relates to devices, methods, and kits for treating cancer using brachytherapy. Electronic x-ray brachytherapy sources that deliver ionizing radiation to a volume of tissue are known in the art. These sources have numerous advantages over traditional radionuclide sources such as iridium-192, including providing physicians the ability to modulate energy emissions and to conduct treatment with comparatively less shielding. However, some clinicians have been reluctant to accept electronic brachytherapy sources in practice. One reason is that these sources can produce unacceptably high radiation doses near surface tissues. A solution to this problem includes the use of a radiation filter to absorb or attenuate low energy x-rays from the source. One apparatus employing aluminum as such a filter is described, by way of useful background information, in U.S. Pat. No. 6,421,416, entitled APPARATUS FOR LOCAL RADIATION THERAPY. Unfortunately, the structural characteristics of such filters can be unacceptable, particularly in applications involving treatment in narrow body cavities.

### SUMMARY OF THE INVENTION

We have discovered that the radial dose function of an electronic x-ray brachytherapy source can be flattened when filtered by transition metals in the fourth row of the periodical table of elements. As a result, a reduction in radiation dose delivered to tissues near the source can be achieved with a comparably smaller penalty in dose rate to tissues farther from the source. Furthermore, we have discovered that certain fourth row transition metals can also provide beneficial structural characteristics to applicators suitable for delivering the source of radiation. According to an embodiment of the invention, the radiation may be delivered in intracavitary tissues or to surface tissues.

In a brachytherapy treatment apparatus embodying the principles of the invention, an applicator is provided that enables an electronic brachytherapy source to be inserted and positioned in a body cavity. Ionizing radiation produced by the source is filtered by the applicator to achieve the novel radial dose function characteristics previously unattained with such sources. According to one embodiment of the invention, the applicator may be formed from titanium to further optimize the size, structural stability, biocompatibility, and imaging compatibility of the applicator, as well as to establish the desired radial dose function. Titanium is a desirable metal as it is compatible under both computed tomography (CT) and magnetic resonance (MR) medical imaging technologies. Other elements in the range of titanium to nickel on the periodic chart can be used, and can be matched to x-ray energy level for desired radial dose function.

In a preferred form of the invention, a brachytherapy device for administering radiation in a narrow body cavity has an applicator body with an electronic x-ray source contained within, and includes means for controlling the source from outside a patient. Preferably the source emits radiation in the range of about 40 keV to about 70 keV. In a distal portion of the applicator body its outside diameter is not greater than about 10 mm, and preferably no greater than about 8 mm or 9 mm. A source lumen is within this distal portion of the applicator body and contains the electronic controllable x-ray source. The source lumen is defined by and surrounded by walls of the distal portion of the applicator body, these walls being of titanium and of a thickness in the range about 0.2 mm to about 0.5 mm. With this structure the applicator achieves a desired, flattened radial dose function for the radiation, to administer a desired dose at about 2 cm from the applicator, particularly for cervical brachytherapy treatment, without overdosing near tissues. In addition, the titanium shell or body in the distal portion provides adequate structural strength in the very thin-shelled distal portion.

More broadly, the invention achieves these advantages with an applicator body formed of a material in the range of titanium to nickel on the periodic chart, i.e. atomic number 22 to 28, with the selected element and shell thickness range matched to the energy of the radiation. These and other objects, advantages and features of the invention will be apparent from the following description of a preferred embodiment, considered along with the accompanying drawings.

Description of the Drawings

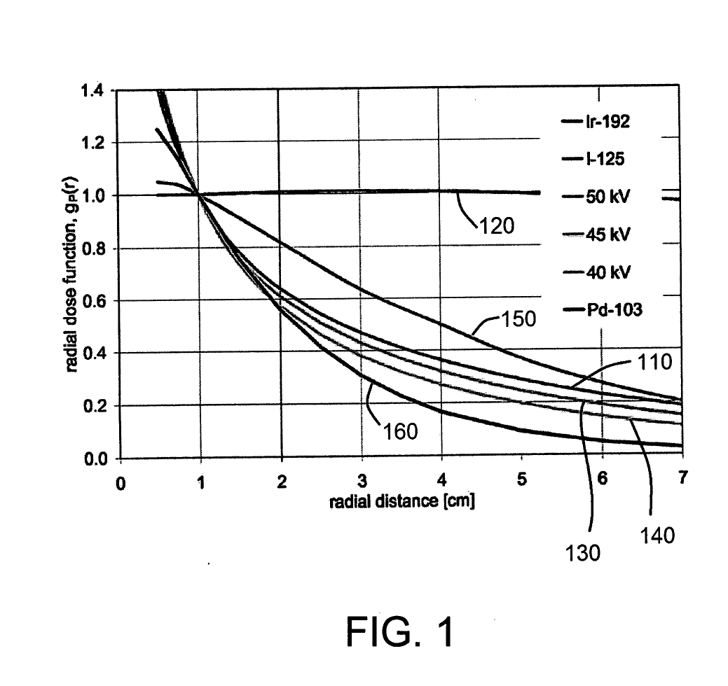

FIG. 1 illustrates the radial dose functions of a 50 kV electronic x-ray source and iridium-192 in water, normalized to 1.0 at a distance of 1 cm.

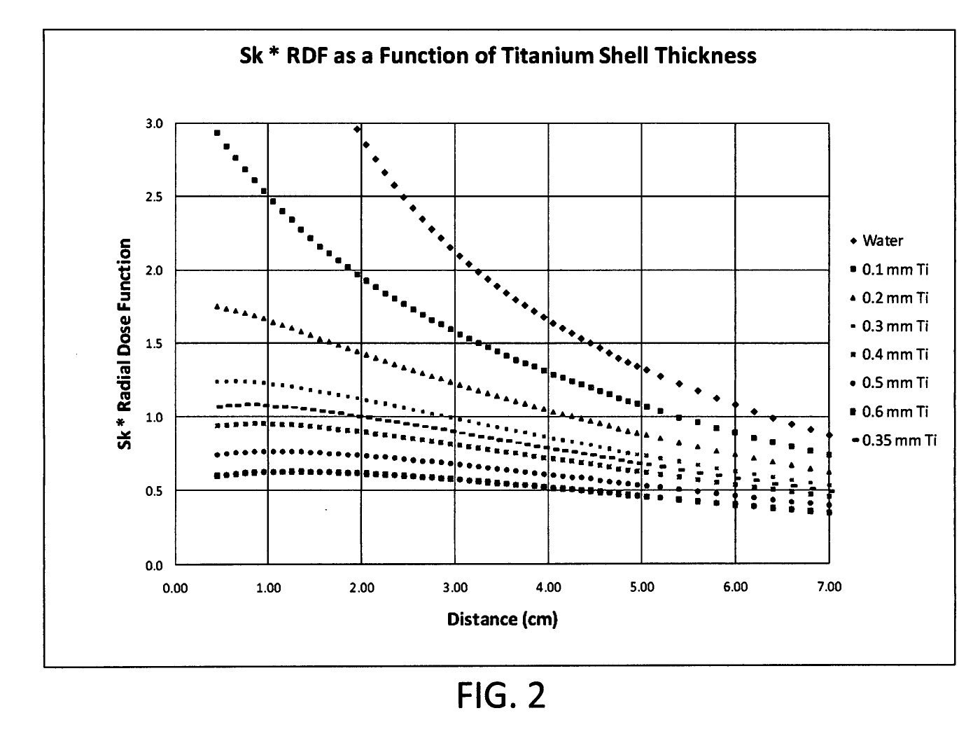

FIG. 2 illustrates a relative measure of the attenuation of a 50 kV electronic x-ray source in water and with different thicknesses of titanium.

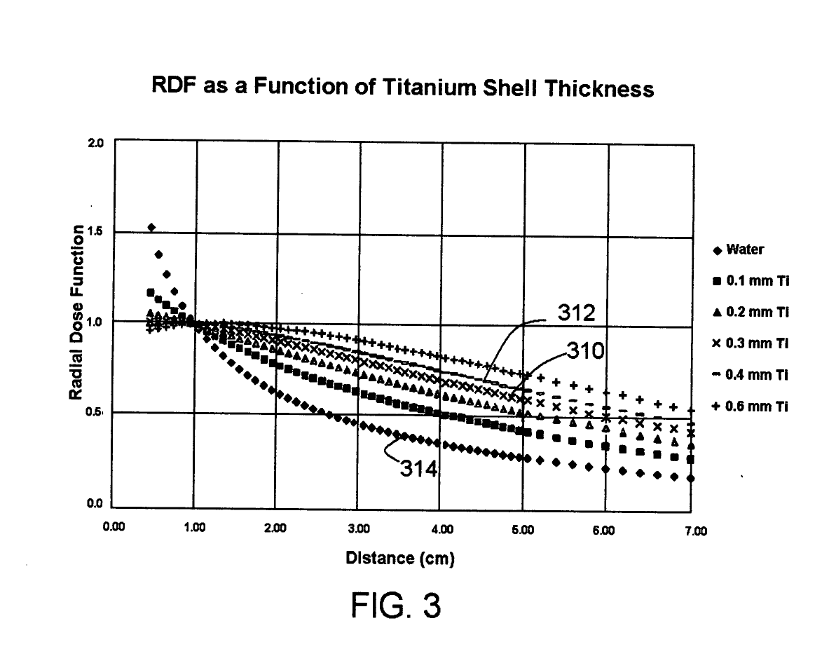

FIG. 3 illustrates the radial dose functions of a 50 kV electronic x-ray source in water and with different thicknesses of titanium, normalized to 1.0 at a distance of 1 cm.

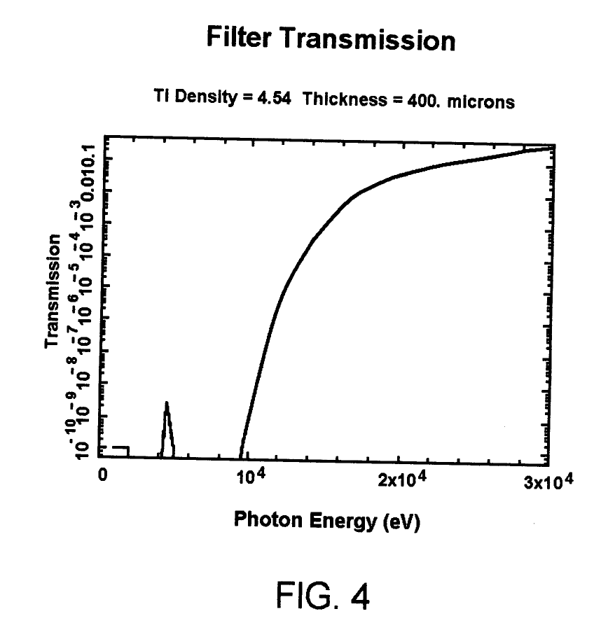

FIG. 4 illustrates a transmission curve for low energy x-rays through 0.4 mm of titanium.

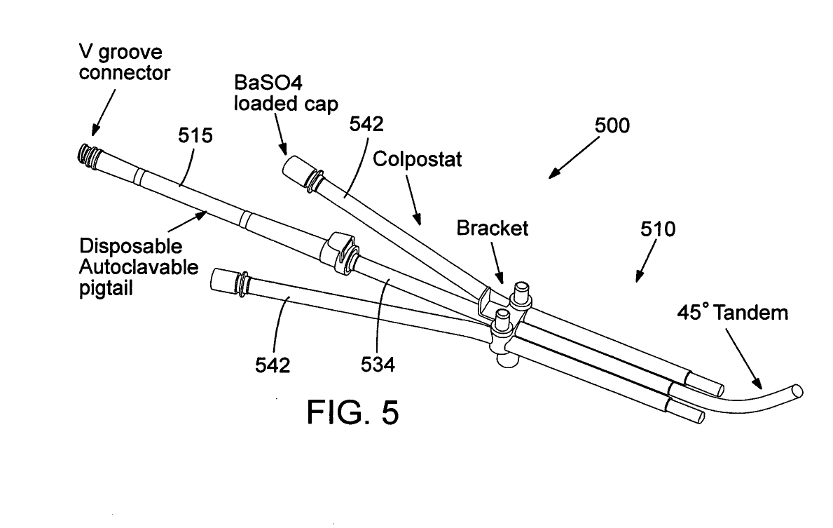

FIG. 5 illustrates an example of a cervical brachytherapy treatment apparatus embodying the principles of the invention.

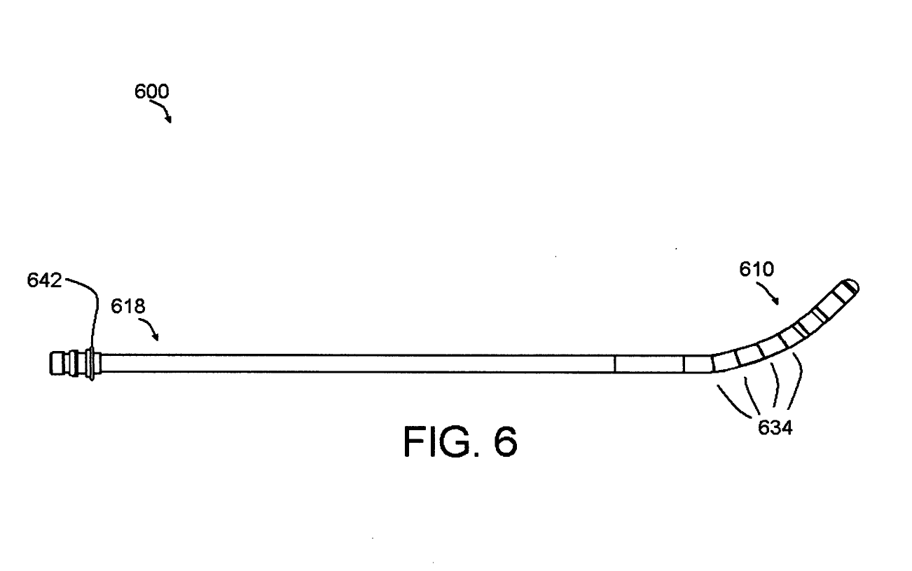

FIG. 6 illustrates an example of a tandem that can be included as part of the cervical brachytherapy treatment apparatus of FIG. 5.

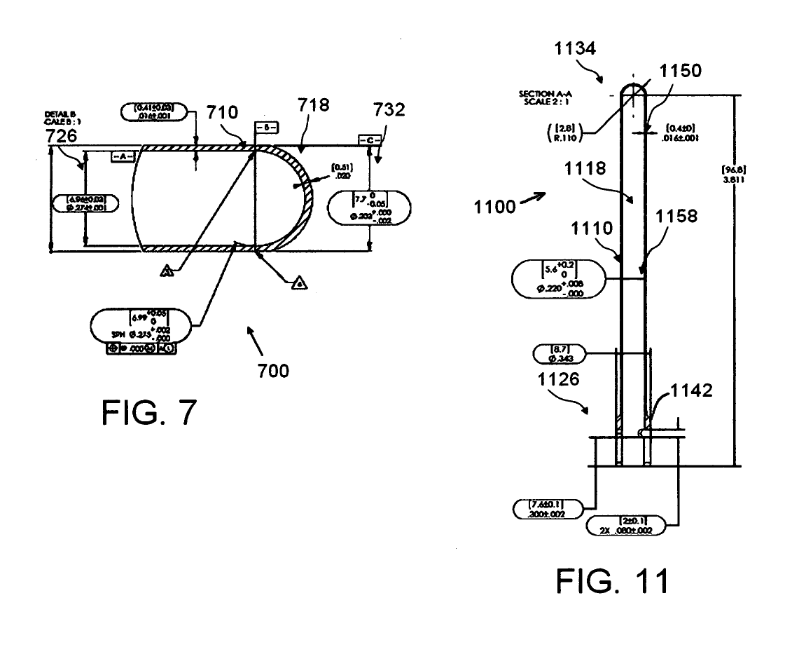

FIG. 7 illustrates a more detailed view of the patient end or distal portion of the tandem shown in FIG. 6.

FIG. 8 illustrates an example of a tandem proximal end and a tandem distal end that can be assembled to form the tandem of FIG. 6.

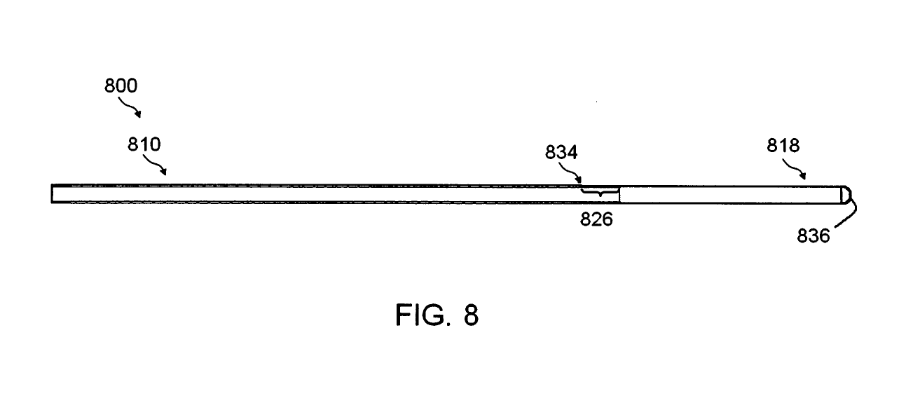

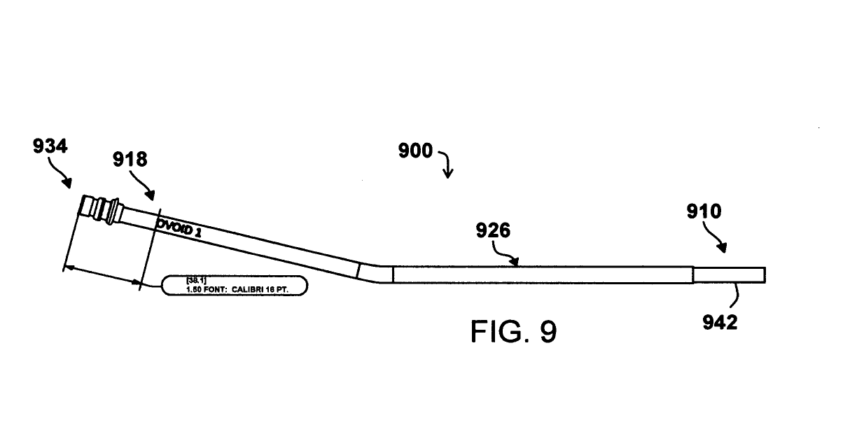

FIG. 9 illustrates an example of a colpostat that can be included as part of the cervical brachytherapy treatment apparatus of FIG. 5.

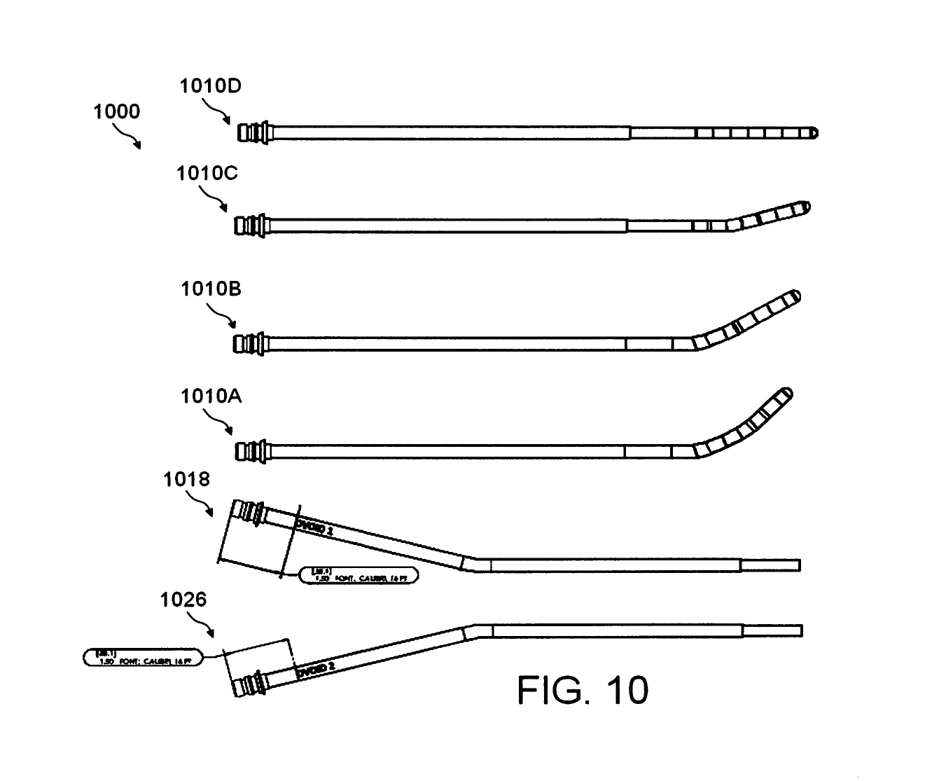

FIG. 10 illustrates an example of a cervical brachytherapy treatment kit embodying the principles of the invention.

FIG. 11 illustrates an example of a spinal brachytherapy treatment apparatus embodying the principles of the invention.

### DESCRIPTION OF PREFERRED EMBODIMENTS

FIG. 1 serves to illustrate why electronic x-ray brachytherapy sources can produce unacceptably high radiation doses near surface tissues. FIG. 1 includes a graph that plots the radial dose function of a 50 kV electronic brachytherapy source in a water bath, represented by curve 110, against the radial dose function of iridium-192 r) in the water bath, which is represented by curve 120. Both curves are normalized such that the source dose rate is 1.0 at 1.0 cm radial distance. Curves for 45 kV and 40 kV are at 130 and 140 in FIG. 1; curves for I-125 and Pd-103 are at 150 and 160, respectively. As is known to one of skill, radial dose functions are unitless. In contrast to ¹⁹²Ir, as radial distance from the source decreases, the radial dose function of the 50 kV electronic brachytherapy source increases, meaning that the electronic brachytherapy source delivers a relatively higher radiation dose at these close distances. This can be problematic when the source is near sensitive tissues such as the vaginal mucosa, for example. Further contrasting these two sources, whereas the radial dose function of ¹⁹²Ir remains relatively consistent as radial distance increases, the radial dose function of the 50 kV electronic brachytherapy source (curve 110) continues to decrease, meaning that relatively longer treatment times are required to deliver a target dose to a treatment medium farther from the source. In the art, “dose rate” is defined as the target dose to a treatment medium per unit time. When the operating voltage of the electronic brachytherapy source is decreased, while there is little change to the radial dose function at close distances, it is more difficult for the radiation to penetrate to greater distances and thus, the radial dose function decrease is more significant.

FIGS. 2-4 illustrate various aspects of an important discovery that was made by the applicants and supports the present invention. The applicants discovered the ability to tune the radial dose function of an electronic brachytherapy source using titanium, which is a transition metal in the fourth row of the periodical table of elements. As a result, the electronic brachytherapy source can deliver radiation to treatment depths of interest at a clinically useful dose rate without overtreating tissues near the source. One skilled in the art is aware that the distances from a source between 0.5 cm and 3.0 cm are the useful clinical ranges of treatment. Further, one skilled in the art knows that the dose rate will decrease with the square of the distance from the source, and that this dose rate reduction is a fundamental law of nature, known as the geometric dose factor. Finally, the historical understanding of brachytherapy is that an additional factor known as the radial dose function will also affect the dose rate with distance, and that the radial dose function is generally associated with the mean energy of the radionuclide used. It is understood that low energy radionuclide sources such as I-125 or Pd-103 have radial dose functions that drop quickly with distance and high energy sources such as Ir-192 or Cs-137 have radial dose functions which decrease very slowly, or even increase slightly over the first three cm of distance. While the radial dose function describes dose over any useful distance, for any given radionuclide source, the clinical utility of the source is historically characterized by comparing doses at two separate distances, such as the maximum dose divided by the dose at the clinically needful depth. As the historically available radionuclide sources are predominantly mono-energetic, it has always been accepted by those in the art that to obtain a clinically acceptable dose ratio at two desired depths, the skilled practitioner will choose a radionuclide with the appropriate energy characteristics. The inventors have discovered that while an unfiltered low energy source, for example a 50 kVp x-ray source, might have a shallow dose penetration, resembling that of I-125 over the clinically useful range of 0.5-3.0 cm, an appropriately filtered source has a deep dose penetration similar to that of Ir-192 in the same depth range. Moreover, by further adjusting the filtration, the same source could have depth dose penetration characteristics similar to radionuclide sources of intermediate energy ranges between I-125 and Ir-192, such as Yb-169. Those skilled in the art may note that filtering low energy Bremsstrahlung x-rays to modify the depth of penetration has been understood for many decades, but this understanding has never been applied to internally placed sources with the specific intent of modulating the radial dose function. Those skilled in the art will also realize that this method of filtration could not generally be usefully applied to a radionuclide as these sources of x-ray energy have energy distributions that do not result in the clinically useful effect that is described here. Those skilled in the art would recognize that geometric dose factor for a single source at a point in space will follow the inverse squares law, whereas a series of sources in a line, a plane or any other spatial distribution will have more complex mathematical descriptions. Those skilled in the art will also note that any such distribution of sources in space can also be described as a superposition of individual sources, and that the fundamental characteristic of any source can be simplified by observing the nature of a single point in space. Therefore, those skilled in the art will know that the dose rate at a few points at different distances from a single source will fully characterize the clinical utility of the source. This characterization is the radial dose function, which is a measurement or model of the dose of a single point in space with the geometrical dose factor removed. Any comparison of dose rates at depth between given sources can be simplified by comparing the radial dose functions of the two sources.

FIG. 2 illustrates a relative measure of the attenuation of a 50 kV electronic x-ray source in a water bath with different thicknesses of titanium and, in the reference case, replacing the titanium with a shell of water. In this example, the dose rate decrease with distance is quantified as the calculated depth-dose curve multiplied by radial distance squared. This graph demonstrates that titanium will moderate the rapid dose fall-off characteristic of the electronic x-ray source. While prior art filtration techniques have achieved a reduction in the amount of dose delivered to tissue close to the source, such achievements have been made at the expense of a larger penalty in dose rate at distances farther away from the source.

In FIG. 3 radial dose functions are presented for the same set of titanium thicknesses as in FIG. 2, but the dose-depth curves are normalized to have a dose rate of 1.0 at 1.0 cm radial distance from the source. These curves demonstrate that the radial dose function flattens as the thickness of titanium is increased. The curves in FIGS. 2 and 3 also demonstrate that a majority of the flattening benefit occurs with a titanium thickness in the range of 0.3 mm (which is represented by curve 310) to 0.4 mm (which is represented by curve 312).

With reference to FIG. 3, following curve 310 out to a distance of about 2.0 cm, titanium increases the radial dose function to about 0.9 from about 0.6 in water only (as represented by curve 314). Thus, about 25% of relative dose deposition is gained at that prescription point. In contrast, at a distance of about 0.5 cm, titanium decreases the radial dose to about 0.95 from 1.6 in water only. Thus, the surface dose has been decreased by 60%. Moving further out to about 3.0 and 4.0 cm, which may represent locations in tissue beyond the treatment volume of interest, the radial dose function decreases to about 0.5-0.6. The curves of FIG. 3 serve to demonstrate that by optimizing titanium thickness in combination with the energy of an electronic brachytherapy source, it is possible to mimic the radial dose function of commonly used radionuclides (such as iridium) to specific prescription points of clinical interest (e.g., 2.0 cm, which is typical in cervical brachytherapy) and then deliver less dose than iridium at greater distances (e.g., beyond 2.0 cm, to sensitive organs such as the rectum and the bladder), while also holding dose to a minimum at near tissues (i.e. 0.5 cm).

FIG. 4 provides some insight as to why titanium might produce this desirable characteristic for electronic x-ray sources. Graph 400 is a transmission curve for x-rays in the energy range from 0 to 30,000 eV (30 keV) through 0.4 mm of titanium. The titanium has no appreciable transmission of very low energy x-rays that would otherwise increase dose at close source distances if left unattenuated. Photon energy gradually increases from 10 keV up to 30 keV, allowing the x-rays to deliver radiation dose to greater depths. As an alternative to titanium, it is recognized that other Row 4 transition metals may produce a similar filtration effect.

Table 1 below lists the increase in treatment time at various radial distances from the source as the thickness of titanium is increased. In the case of a prescription distance of 2.0 cm, this table demonstrates that a thickness of titanium in the range of 0.3-0.4 mm would increase the treatment time by about 2.9x, which is within clinically acceptable levels.

TABLE 1

| | |
|---|---|
| Titanium | Radial Distance from the Source (cm) |

| Thickness (mm) | 0.5 | 1.0 | 1.5 | 2.0 | 2.5 |
|---|---|---|---|---|---|
| 0.00 | 1.00 | 1.00 | 1.00 | 1.00 | 1.00 |
| 0.10 | 2.38 | 1.86 | 1.63 | 1.49 | 1.41 |
| 0.20 | 3.93 | 2.80 | 2.31 | 2.03 | 1.85 |
| 0.30 | 5.57 | 3.81 | 3.03 | 2.60 | 2.34 |
| 0.35 | 6.43 | 4.34 | 3.42 | 2.91 | 2.60 |
| 0.40 | 7.34 | 4.90 | 3.83 | 3.24 | 2.87 |
| 0.50 | 9.28 | 6.11 | 4.72 | 3.95 | 3.47 |
| 0.60 | 11.44 | 7.45 | 5.70 | 4.73 | 4.12 |

### Application to Cervical Brachytherapy

By way of useful background, invasive cancer of the cervix is the major cause of death from gynecologic cancer worldwide, with almost half a million cases diagnosed each year. Reported incidence rates in developing countries are much higher than those in developed countries (about 80% vs. 20%). The curative treatment of cervical cancer with primary radiation usually includes a combination of external pelvic irradiation and intrauterine/intravaginal brachytherapy. The goal of radiation is to eliminate cancer in the cervix, paracervical tissues, and regional lymph nodes. It is recognized that the use of brachytherapy for the cervical area in addition to external radiation to pelvic and paraaortal regions is beneficial to the patient. Furthermore, it is critical to limit surface dose intrauterine, on vaginal mucosa, rectosigmoid, bladder and small bowel to currently accepted levels.

FIG. 5 illustrates an example of a brachytherapy treatment apparatus 500 that can take advantage of the discoveries described herein for the treatment of cervical cancers. The apparatus includes a multi-channel applicator 510 with various channels through which an electronic brachytherapy source can be deployed into various positions within the treatment volume. The electronic brachytherapy device can include an x-ray catheter carrying an x-ray tube, not specifically shown but within a pigtail 515 and a tandem 534 shown coupled thereto. According to certain embodiments of the invention, the x-ray tube produces an x-ray spectrum with maximum energies in the range of 30-70 keV depending upon operating voltage, and the catheter carrying the source may have a diameter of about 5.4 mm. By way of useful background, x-ray tubes producing energy in the mid-point of this range and arrangements for cooling such x-ray tubes by pumping water through the catheter have been described in the prior art (see, for example, U.S. Pat. No. 6,319,188, titled VASCULAR X-RAY PROBE; and U.S. Pat. No. 7,127,033, titled MINIATURE X-RAY TUBE COOLING SYSTEM. One of skill will recognize other electronic brachytherapy sources that can be employed in conjunction with the invention.

The applicator includes the tandem 534 to facilitate radiation treatment of the uterus. The applicator also includes a pair of lateral tubes or colpostats 542, to the ends which can be attached ovoids (not shown). This apparatus in FIG. 5 facilitates treatment of the right and left vaginal fornices, the cervix, and the parametrium or cervical stroma. As illustrated in FIG. 5, the tandem 534 is the central or medial channel flanked on both sides by the colpostats 542. According to one embodiment of the invention, both the colpostats and the tandem can include a layer or wall thickness of titanium as described herein to take advantage of the desirable x-ray transmission characteristics, the structural advantages, and the tertiary advantages described herein. The pigtail 515, shown connected to the tandem, can be coupled to each of the devices 534, 542 to insert the catheter therein. The proximal end of the pigtail can be connected to a control system controlling radiation and positions of the source.

FIG. 6 illustrates an example of a tandem 600 that can be included as part of the cervical brachytherapy treatment apparatus of FIG. 5. The tandem includes a patient or distal end 610, a proximal end 618, and an inner source lumen within the tandem through which an electronic brachytherapy source can be deployed into the uterine canal of a patient.

FIG. 7 is a more detailed view of a patient or distal end 700 that can be included as part of the tandem 600. The distal end includes a wall 710 that, in accordance with an embodiment of the invention, can be constructed with a thickness of titanium that optimizes both the structural stability of the tandem and the x-ray transmission characteristics when radiation treatment using an electronic brachytherapy source is delivered from within the device. For example, the titanium of the wall can have a thickness in the range of 0.3 mm to 0.45 mm, or 0.37 mm-0.44 mm, which is particularly suitable in combination with an electronic brachytherapy source operating at about 50 kV.

The distal end 700 also included a dome tip 718. In accordance with certain embodiments of the invention, retracting the radiation source through the channel produces an air pocket between the source and the dome tip. One example of such a source is such as a source catheter with x-ray tube. Thus, a larger amount of radiation can be transmitted through the dome tip of the tandem than predicted by conventional radiation treatment planning systems. By increasing the thickness of titanium at the dome tip, the amount of x-ray attenuation can be increased at this location, thereby allowing for a more even distribution of radiation delivered at the distal end. In such embodiments, the assembled distal end of the tandem is of a nonuniform wall thickness. For example, the dome tip may have a wall thickness in the range of 0.48 mm-0.54 mm, which is particularly suitable in combination with an electronic brachytherapy source operating at about 50 kV.

With respect to structural stability, noting the 45° curvature illustrated in FIG. 6, the tandem receives force from patient tissues upon insertion and must have enough structural strength so that it does not change shape or crimp during use. It has been found that a particular range of wall thicknesses will not only satisfy the aforementioned desirable x-ray attenuation properties, but also enable the tandem to have adequate strength to maintain its shape, have an inner diameter 726 large enough for an electronic brachytherapy source, which can be larger in diameter than a traditional radioisotope source, to be positioned around the curvature and into the distal end without sustaining damage and to achieve all of this without increasing the tandem to a clinically unacceptable overall diameter. For example, the illustrative wall thickness in the range of 0.3 to 0.4 mm, or 0.37 mm-0.44 mm may allow for an inner diameter in the range of 6.9 mm-7.0 mm and an outer diameter in the range of 7.8 mm-8.1 mm, all of which may satisfy the aforementioned desirable characteristics for tandems with significant curvatures (e.g., 30° and 45°). In embodiments involving tandems of lesser curvatures (e.g., 0° and 15°) and/or electronic brachytherapy sources where susceptibility to damage during positioning is of less concern, the inner and outer diameters may be smaller than the exemplary ranges provided herein above.

Again referencing FIG. 6, the wall of the proximal end 618 may be constructed with a reinforced section to provide additional structural strength to the applicator. Such strength may be necessary for attaching the colpostats and the tandem to a bracket or other structural support. Thus, the proximal end wall may be constructed with an increased thickness of titanium relative to the distal end wall. For example, the reinforced section may have a wall thickness in the range of 0.8 mm-0.98 mm, which provides a larger outside diameter, not critical in this proximal section so long as internal diameter is maintained.

As illustrated in FIG. 6, the tandem distal end can be laser marked with a plurality of markings 634 to aid in the accurate placement of a cervical stopper (not shown in FIG. 6) and positioning of the applicator in the uterine canal. The markings can be rings and can be spaced 1 cm apart to simulate a metric ruler. The tandem proximal end can also include a hub or connector 642 if deployment of the electronic brachytherapy source requires mating the applicator with a source controller. The hub can be a flexible, V-groove connector made of polysulfone/silicone.

FIG. 8 illustrates the tandem of FIG. 6 as two assembled components: a tandem proximal end 810 and a tandem distal end 818. The tandem proximal end 810 is constructed with a distal portion 826 having a thinner wall of titanium than the rest of the proximal end. During the manufacturing process, the tandem distal end 818 can be slid over the proximal end portion 810 until both tubes meet at junction 834. The components and dome tip (not shown) can be laser welded together. The proximal end of the assembled device can then be bent until the desirable curvature is achieved.

A tandem of lesser curvature (e.g., 0° or 15° curvature) can be manufactured from a single tube, as opposed to assembly from two manufactured components. The tube can be manufactured with a thicker wall (e.g., 0.8 mm-0.98 mm of titanium) and the proximal end can then be ground down to the thinner wall (e.g., 0.37 mm-0.44 mm of titanium). We have found that this approach removes an additional processing step and produces a tandem possessing greater structural strength.

FIG. 9 illustrates an example of a colpostat 900 that can be included as part of the cervical brachytherapy treatment apparatus of FIG. 5. The colpostat includes a patient or distal end 910, a proximal end 918, and an inner source lumen within the colpostat through which an electronic brachytherapy source can be deployed into the vaginal canal of a patient. The colpostat proximal end can also include a hub or connector 934.

The distal end 910 of the colpostat includes a wall 942 that, in accordance with an embodiment of the invention, can be constructed with a thickness of titanium that optimizes structural stability and x-ray transmission characteristics when radiation treatment using an electronic brachytherapy source is delivered from within the device. For example, a wall thickness in the range of 0.37 mm-0.44 mm, an inner diameter in the range of 6.9 mm-7.0 mm, and an outer diameter in the range of 7.8 mm-8.1 mm may satisfy the aforementioned desirable characteristics and which is non-critical so long as internal diameter is maintained.

The length of the colpostat distal end with this particular wall thickness can be much smaller with respect to the area of the tandem distal end, as a fewer number of source dwell positions may be needed to deliver radiation. For example, according to one illustrative treatment plan, the tandem distal end may support 15 dwell positions by accommodating a 7.5 cm pullback distance; the colpostat distal end may support 2 dwell positions. Any number of dwell positions, and any length of distal end, may be used as needed. A dwell position refers to the location from which source radiation is delivered over a time interval so that target coverage and organ at risk avoidance are optimized in accordance with treatment planning.

The wall of the proximal end 918 may be constructed with a reinforced section to provide additional structural strength to the applicator. Such strength may be necessary for attaching the colpostats and the tandem to a bracket or other structural support. Thus, the proximal end wall may be constructed with an increased thickness of titanium relative to the distal end wall. For example, the reinforced section may have a wall thickness in the range of 0.8 mm-0.98 mm, for a larger-diameter proximal section, which is non-critical so long as internal diameter is maintained.

FIG. 10 illustrates a cervical applicator kit 1000 embodying the principles of the invention. The kit contains a plurality of tandems 1010A-1010D, a left colpostat 1018, and a right colpostat 1026. Each tandem is formed with a different curvature to enable the clinician to match the geometry of the tandem with the shape and size of patient anatomy. For example, a 45° curvature tandem 1010A, a 30° curvature tandem 1010B, a 15° curvature tandem 1010C, and a straight (0°) tandem 1010D may be provided in such a kit. Each tandem and colpostat in the kit may be constructed using a material or combination of materials that provide desired x-ray transmission and structural characteristics in accordance with the invention.

The kit 1000 can include other components for cervical brachytherapy treatment (not illustrated in FIG. 10) with a tandem and colpostats applicator such as, but not necessarily limited to, a set of guidetube assemblies, each of which enables a tandem or colpostat to be attached to an electronic brachytherapy source controller; a bracket for fixing the relative positions of the tandem and colpostats; a cervical stop or stopper that can be attached to the tandem and aids in preventing accidental perforation of the uterus; and a set of ovoids (Hantel Technologies, Hayward, Calif.), each of which can be mounted to the distal end of a colpostat and provide spacing with respect to surrounding tissues during use. The set can include ovoids of different sizes (e.g., 2.0 cm, 2.5 cm, and 3.0 cm) and with various shielding configurations (e.g., right shielding, left shielding, right and left shielding, no shielding) so as to enable treatment of cancers of different symmetries.

### Treatment Method

The invention can be illustrated by the following treatment method: Delivering a therapeutic dose of radiation at a nominal dose rate of about 0.3 Gy per minute at a distance of about 2 centimeters from the surface of the electronic brachytherapy source and a dose rate in the range of 1.75-5.0 Gy per minute at a distance of about 0.5 centimeter from the surface of the electronic brachytherapy source. The range of dose rates is subject to the length of the linear train of source positions used and also to the thickness of the filtration material chosen. This range results in a clinical dose ratio at two depths, 0.5 cm and 2.0 cm, ranging from 5.8-16.7 (depending on single emission point or series of positions). For comparison, an unfiltered 50 kV source has a dose ratio between these two points of about 42 (single position), where a commonly used Ir-192 source has a ratio of about 5-16.6.

### Application to Spinal Brachytherapy

FIG. 11 illustrates an example of an interstitial applicator or sheath 1110 that can be used in combination with an electronic brachytherapy source to achieve the advantages described herein for the treatment of spinal cancers. The applicator includes a inner source lumen 1118 that enables the electronic brachytherapy source to be inserted via a proximal end and 1126 positioned within the treatment volume at a patient or distal end 1134. The proximal end can include a connector 1142 that enables the applicator to be connected to a source controller (not shown).

According to one embodiment of the invention, a distal wall 1150 may be constructed with a thickness of titanium in the range of about 0.4 mm (more broadly, 0.35 to 0.45), which is of particular interest to electronic brachytherapy sources operating at about 50 keV, or abut 45 keV to about 55 keV. The applicator can have an inner diameter 1158 in the range of about 5.5 mm-5.8 mm to enable an x-ray catheter with x-ray tube to be deployed through the lumen.

It should be understood that for certain applications the devices described herein or similar tubular applicator devices of titanium or other referenced metals could be used within an outer cylinder or sheath, especially where small diameter is not critical, for purposes of further filtration, structural stability or other reasons. Further, the device could be used within a balloon for treatment of spherical or ellipsoid shapes such as breast lumpectomy cavities or excised brain glioma cancers.

While certain embodiments of the invention have been described with particular reference to titanium, we recognize that other transition metals and/or alloys in the fourth row of the periodic table of elements may be suitable for attenuating x-rays with energies below 20 keV without significant attenuation above that value. In particular, titanium through nickel, possibly in various alloy combinations that include stainless steel, may provide both x-ray transmission and certain structural characteristics of interest to this invention. As ferromagnetic elements, iron, nickel, cobalt, and non-18/10 stainless steels may be less desirable due to non-magnetic resonance imaging compatibility. Copper and zinc may be less desirable due to biocompatibility concerns.

References to titanium herein as the wall material are intended to include titanium alloys containing titanium in amounts sufficient to achieve the effects described. Preferably, for the small-diameter instruments described, titanium content is at least 80%, and preferably at least 90%. Many alloys are common, and may include aluminum, vanadium, nickel, molybdenum, chromium, zirconium, zinc or other metals. References to other metals as wall materials are to be considered similarly to include alloys.

While certain ranges of wall thicknesses, inner diameters, and outer diameters are provided in this disclosure, these ranges may be optimized for specific materials, sources, and source voltages. Additional factors may be under consideration as well. Thus, deviations from such illustrative ranges are possible. As a general rule of thumb, we recognize that to achieve comparable x-ray filtration characteristics with a higher source voltage and a larger size electronic brachytherapy source, wall thicknesses must be increased and in some cases, the selection of transition metal/alloy may shift higher with respect to the location on the fourth row of the periodic table. We have identified that stainless steels, more particularly 300 series stainless steels due to their magnetic resonance imaging-compatibility, represent a suitable alternative to titanium, in particular, either in combination with an electronic brachytherapy source operating at around 70 keV at the comparable wall thicknesses discussed in this disclosure; or for use in an applicator (e.g., spinal) where lesser wall thickness (e.g., around 0.14 mm) is desired to reduce the size of the applicator, yet structural strength and filtration are also still important properties. Such combinations are identified as providing additional, concrete alternatives to achieving desirable x-ray filtration and structural characteristics suitable for delivering the source of radiation in accordance with the invention.

What is claimed is:

1. A brachytherapy applicator device for administering radiation from an electronic source particularly in a narrow body cavity, comprising:
an applicator body with an electronic source contained within the body, with means for controlling the source from outside a patient, the source emitting radiation in the energy range of about 30 keV to about 70 keV,
the applicator body having an outside diameter, in a distal portion for insertion into a body cavity, not greater than about 10 mm, and having a source lumen in the applicator with the electronic x-ray source contained therein,
the applicator body having walls surrounding in said distal portion the source lumen, the walls being of titanium, of a thickness in the range of about 0.2 mm to about 0.6 mm.

2. The applicator of claim 1, wherein the wall thickness is in the range of about 0.3 mm to 0.45 mm.

3. The applicator of claim 1, wherein the energy range of the radiation from the source is about 45 Kv to about 55 Kv, and the thickness of the walls is about 0.3 mm to 0.45 mm.

4. The applicator of claim 1, wherein the outer diameter of said distal portion of the applicator body is not greater than about 8 mm.

5. The applicator of claim 1, wherein the outer diameter of said distal portion of the applicator body is not greater than about 7 mm.

6. The applicator of claim 1, wherein the walls of titanium have a titanium content of at least about 80%.

7. The applicator of claim 1, wherein the walls of titanium have a titanium content of at least about 90%.

8. The applicator of claim 1, wherein the applicator device is a tandem.

9. The applicator of claim 1, wherein the applicator device is a colpostat.

10. The applicator of claim 1, wherein the applicator device is an interstitial applicator.

11. A brachytherapy applicator device for administering radiation from an electronic source particularly in a narrow body cavity, comprising:
an applicator body with an electronic source contained within the body, with means for controlling the source from outside a patient, the source emitting radiation in the energy range of about 30 keV to about 70 keV,
the applicator body having an outside diameter, in a distal portion for insertion into a body cavity, not greater than about 10 mm, and having a source lumen in the applicator with the electronic x-ray source contained therein,
the applicator body having walls surrounding in said distal portion the source lumen, the walls being of a transition metal in the fourth row of the periodic table of elements in the range of titanium through nickel, or alloys thereof, of a thickness in the range of about 0.2 mm to about 0.6 mm.

12. The applicator of claim 11, wherein the radiation is in the energy range of about 60 keV to 70 keV, and the metal is 300-series stainless steel.

13. The applicator of claim 11, wherein the radiation is in an energy range of about 45 keV to 55 keV, the metal is titanium, and the wall thickness is in the range of about 0.3 mm to 0.45 mm.

14. A method for administering brachytherapy within the cervix of a patient, comprising:
delivering a therapeutic dose of radiation from an electronic radiation source extending into the cervix, at a nominal dose rate of about 0.3 Gy per minute at a distance of about 2 centimeters from the surface of the electronic brachytherapy source and at a dose rate in the range of about 1.75 to 5.0 Gy per minute at a distance of about 0.5 centimeter from the surface of the electronic brachytherapy source.

15. The method of claim 14, wherein the electronic brachytherapy source is within the distal end of an applicator device formed of titanium, with a wall thickness surrounding the brachytherapy source of about 0.3 to 0.45 mm, the electronic brachytherapy source emitting radiation at an energy level of about 45 keV to 55 keV.
